SEQUENCE LISTING (7 sequences)

SEQ ID NO: 1 - skipped

SEQ ID NO: 2 - protein, 18 aa, synthetic construct
SRALKIFLATRKDRSPWH

SEQ ID NO: 3 - protein, 18 aa, synthetic construct
HKSRARLIFHEGPCGVRR

SEQ ID NO: 4 - protein, 10 aa, synthetic construct
SRALLIFQKI

SEQ ID NO: 5 - protein, 18 aa, synthetic construct
VSRATRIFKLSQLRGQCA

SEQ ID NO: 6 - protein, 7 aa, synthetic construct
SRAXXIF

SEQ ID NO: 7 - protein, 18 aa, synthetic construct
HKSRALLIFQKIMWLRRQ

(19) United States
(12) Patent Application Publication
(10) Pub. No.: US 2026/0184742 A1
(43) Pub. Date: Jul. 2, 2026

(54) PEPTIDES

(71) Applicant: CAROCELL BIO LIMITED, London (GB)

(72) Inventor: Michael John Davies, London (GB)

(73) Assignee: CAROCELL BIO LIMITED, London (GB)

(21) Appl. No.: 19/130,306

(22) PCT Filed: Nov. 16, 2023
(86) PCT No.: PCT/GB2023/053019

§ 371 Date: May 15, 2025

(30) Foreign Application Priority Data
Nov. 16, 2022 (GB) 2217153.2

(51) Int. Cl. C07K 7/08; A61K 38/00; A61K 45/06; A61P 29/00

(52) U.S. Cl. CPC C07K 7/08; A61K 45/06; A61P 29/00; A61K 38/00

(57) ABSTRACT

There is described a compound comprising a sequence of at least 3 amino acids wherein the sequence of 3 amino acids comprises the sequence Ser-Arg-Ala; and variants and derivatives thereof; provided that the amino acid is not: His-Lys-Ser-Arg-Ala-Leu-Leu-Ile-Phe-Gln-Lys-Ile-Met-Trp-Leu-Arg-Arg-Gln.

### FIELD OF THE INVENTION

The present invention relates to peptides which are MAP kinase inhibitors and uses thereof.

More particularly, the invention relates to novel synthetic peptides which are useful as inhibitors of p38α mitogen-activated protein kinase (p38α MAP kinase or MAPK14). Therefore, the invention relates to uses of the novel peptides, methods of treatment related thereto and methods of making such novel peptides.

### BACKGROUND OF THE INVENTION

p38α MAP kinase is involved in the regulation of inflammatory cell signals and plays a central role in the regulation of pro-inflammatory cytokine production.

Therefore, the novel peptides of the invention have potential for the treatment of inflammatory or autoimmune diseases, including cancer.

The p38 kinase regulates the production of key inflammatory mediators by cells of the innate immune system, including TNFalpha, IL-1beta, and COX-2.

MAP Kinase p38α plays a central role in the inflammation signalling cascade and therefore its inhibition is believed to have a beneficial role in a range of human and animal disease indications, especially inflammatory or autoimmune disorders and cancer. These include, but are not limited to, inflammatory disorders of the skin such as atopic dermatitis, psoriasis, acne vulgaris. Activation of p38α MAP kinase may also play a fundamental role in other disorders involving inflammation including, constrictive obstructive pulmonary disease, asthma, inflammatory bowel disease, atherosclerosis, cancer, burns, scarring and rheumatoid arthritis.

European Patent No. 3033094 describes a compound comprising the amino acid sequence HKSRALLIFQKIMWLRRQ, which binds to p38α MAP kinase. The compound is useful in binding to and inhibiting p38α MAP kinase and is useful as a research tool, in drug discovery and medicine, particularly for treating inflammatory or autoimmune disorders.

### SUMMARY OF THE INVENTION

Thus, according to a first aspect of the invention there is provided a compound comprising a sequence of at least amino acids wherein the sequence of 3 amino acids comprises the sequence Ser-Arg-Ala;
and variants and derivatives thereof,
provided that the amino acid is not:

His-Lys-Ser-Arg-Ala-Leu-Leu-Ile-Phe-Gln-Lys-Ile-
Met-Trp-Leu-Arg-Arg-Gln.

According to a further aspect of the invention there is provided a compound comprising a sequence of at least 7 amino acids wherein the sequence of amino acids comprises Ser-Arg-Ala-A-B-Ile-Phe, wherein A and B, which may be the same or different, may be any amino acid moiety;
and variants and derivatives thereof;
provided that the amino acid is not:

His-Lys-Ser-Arg-Ala-Leu-Leu-Ile-Phe-Gln-Lys-Ile-
Met-Trp-Leu-Arg-Arg-Gln.

According to a further aspect of the invention the compound may be selected from the group comprising the amino acids:

Ser-Arg-Ala-Leu-Lys-Ile-Phe-Leu-Ala-Thr-Arg-Lys-
Asp-Arg-Ser-Pro-Trp-His;
His-Lys-Ser-Arg-Ala-Arg-Leu-Ile-Phe-His-Glu-Gly-
Pro-Cys-Gly-Val-Arg-Arg;
Ser-Arg-Ala-Leu-Leu-Ile-Phe-Gln-Lys-Ile;
and
Val-Ser-Arg-Ala-Thr-Arg-Ile-Phe-Lys-Leu-Ser-Gln-
Leu-Arg-Gly-Gln-Cys-Ala;

and variants and derivatives thereof.

The IUB/IUPAC one letter code for amino acids is used throughout the specification unless otherwise noted, and the amino acid sequence is given in the conventional N- to C-terminal direction. Typically, the amino acids are L-amino acids, some of them may be D-amino acids.

The peptides of the invention may comprise variants, e.g. larger peptides with additional amino acids, in which the peptides of the invention form only part of the variant peptide. In the variant peptides amino acids are typically naturally occurring amino acids, which include the 20 amino acids that are encoded by the genetic code, and also include other natural amino acids such as hydroxyproline, selenomethionine, and carnitine. However, one or more of the altered amino acids may be non-natural amino acids, such as allylalanine and diphenylalanine.

The novel peptides of the present invention described herein are useful in binding to and inhibiting p38α MAP kinase, and are useful as research tools, in drug discovery and in human and veterinary medicine, particularly for treating or preventing proliferative disorders (e.g. cancer), autoimmune disorders, inflammatory-disorders, metabolic disorders, and neurological disorders. Such disorders may exist in a range of mammals, including man and dogs. Advantageously, compounds are provided that are selective for p38α MAP kinase and do not substantially bind to, or inhibit, other protein kinases.

The variants and derivatives herein described may comprise any of the [3], [7], [10] or [18] amino acid sequences described, as contiguous amino acid sequences.

Typically when the compound consists of a peptide it has a molecular weight of less than 30,000 daltons, preferably less than 25,000 daltons, more preferably less than 20,000 daltons. Typically, when the compound comprises a peptide portion linked to another moiety as discussed below, the compound may typically have a molecular weight of between 10,000 daltons and 100,000 daltons, such as between 20,000 daltons and 80,000 daltons, for example between 20,000 daltons and 50,000 daltons.

The amino acid sequences of the compounds are peptides, by which term we include compounds that have amino acid residues (H-Cα-[side chain]) but which may be joined by peptide (—CO—NH—) or non-peptide linkages.

Peptides may be synthesised by the Fmoc-polyamide mode of solid-phase peptide synthesis as disclosed by Lu et al (1981) J. Org. Chem. 46, 3433, incorporated herein by reference, and references therein. Reagents for peptide synthesis are readily commercially available.

Purification of the compounds may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

According to one aspect of the invention the compound comprises the amino acids Ser-Arg-Ala-Leu-Lys-Ile-Phe-Leu-Ala-Thr-Arg-Lys-Asp-Arg-Ser-Pro-Trp-His; and variants and derivatives thereof.

According to another aspect of the invention the compound comprises the amino acids His-Lys-Ser-Arg-Ala-Arg-Leu-Ile-Phe-His-Glu-Gly-Pro-Cys-Gly-Val-Arg-Arg; and variants and derivatives thereof.

According to another aspect of the invention the compound comprises the amino acids Ser-Arg-Ala-Leu-Leu-Ile-Phe-Gln-Lys-Ile; and variants and derivatives thereof.

According to another aspect of the invention the compound comprises the amino acids Val-Ser-Arg-Ala-Thr-Arg-Ile-Phe-Lys-Leu-Ser-Gln-Leu-Arg-Gly-Gln-Cys-Ala; and variants and derivatives thereof.

The compounds of the invention shall include variants of said amino acid sequences or of said part thereof in which from 1 to 5 amino acids have been altered, provided that the amino acid sequences comprise a sequence of 3 amino acids Ser-Arg-Ala or a sequence of 7 amino acids wherein the sequence of amino acids comprises Ser-Arg-Ala-A-B-Ile-Phe, wherein A and B, are as herein defined; provided that the amino acid is not:

His-Lys-Ser-Arg-Ala-Leu-Leu-Ile-Phe-Gln-Lys-Ile-
Met-Trp-Leu-Arg-Arg-Gln.

wherein the compound binds to p38α MAP kinase.

The compounds of the invention may be N-acetylated, some or all of the amino acids in the peptide may be N-acetylated.

The compound may contain at least one D-amino acid residue, such as 1, 2 or 3 or 4 or 5 or 6 or 7 or 8 D-amino acids. Typically, the compounds may contain 0, 1, 2 or 3 D-amino acids. The presence of D-amino acids in the compounds may be useful in preventing degradation of the compounds by proteases. Other methods for making peptides resistant to proteolytic degradation include blocking the N- and/or C-terminal amino acid residues. Thus, the N- and/or C-terminal amino acid residues are blocked. Suitable blocking methods include acetylation of the N-terminus or incorporating a pyroglutamate residue at the N-terminus.

Although it is preferred if the compound is a peptide as defined, the compound may comprise a variant as herein defined wherein the peptide of the invention is joined to another moiety. Convenient moieties to which the peptide may be joined include polyethylene glycol (PEG) and peptide sequences, such as TAT and antennapedia which enhance delivery to cells.

PEGylation is a method well known to those skilled in the art wherein a (peptide or other compound) is modified such that one or more polyethylene glycol (PEG) molecules are covalently attached to the side chain of one or more amino acids. It is one of the most important molecule altering structural chemistry techniques (MASC). Other MASC techniques may be used; such techniques may improve the pharmacodynamic properties of a compound, for example extending its serum half-life in vivo. A PEG-peptide conjugate is formed by first activating the PEG moiety so that it will react with, and couple to, the compound described. PEG moieties vary considerably in molecular weight and conformation, with the early moieties (monofunctional PEGs; mPEGs) being linear with molecular weights of 12 kDa or less, and later moieties being of increased molecular weights. PEG2, a recent innovation in PEG technology, involves the coupling of a 30 kDa (or less) mPEG to a lysine amino acid (although PEGylation can be extended to the addition of PEG to other amino acids) that is further reacted to form a branched structure that behaves like a linear mPEG of much greater molecular weight.

The potential advantages of PEGylation of the compound include reduced renal clearance which, for some products, results in a more sustained adsorption after subcutaneous administration as well as restricted distribution, possibly leading to a more constant and sustained plasma concentrations and hence an increase in clinical effectiveness. Further potential advantages include reduced immunogenicity of the therapeutic compound, and lower toxicity.

There are a number of different approaches to the design and synthesis of variant peptide compounds that do not contain amide bonds. In one approach, one or more amide bonds may be replaced in an essentially isosteric manner by a variety of chemical functional groups.

Retro-inverso peptidomimetics, in which the peptide bonds are reversed, can be synthesised by methods known in the art. This approach involves making pseudopeptides containing changes involving the backbone, and not the orientation of side chains. Retro-inverse peptides, which contain NH—CO bonds instead of CO—NH peptide bonds, are more resistant to proteolysis.

It may be advantageous to introduce a cyclic moiety into a variant peptide-based framework. The cyclic moiety restricts the conformational space of the peptide structure and this may lead to an increased affinity of the peptide for p38α. MAP kinase. An added advantage of this strategy is that the introduction of a cyclic moiety into a peptide may also result in the peptide having a diminished sensitivity to cellular peptidases.

The compounds of the invention are useful, inter alia, because they are capable of binding to p38α MAP kinase. Conveniently, the enzyme may be fused to a polypeptide sequence, such as a GST, that allows for its immobilisation on a solid substrate.

Moreover, the compounds of the invention are useful, inter alia, because they inhibit p38α MAP kinase. Inhibition of p38α MAP kinase may be determined as follows. Myelin basic protein (MBP) is a substrate of p38αMAP kinase and is phosphorylated in the presence of this enzyme and ATP. The phosphorylation event may be monitored using antibodies which recognise the phosphorylated serine or threonine residue, or by measuring the incorporation of radioactively labelled phosphate into the protein. Inhibition is measured by a decrease in the phosphorylation of MBP. The compounds of the invention may inhibit the activation of p38α MAP kinase by MKK6.

A suitable way of determining binding to p38α MAP kinase is to use phage display techniques. DNA encoding the peptide leads can be cloned into the M13 gplll phagemid vector, transformed into E. coli TG1 cells and plated on 2% glucose, 2×TY, 100 μg/ml ampicillin plates. The colonies are grown for the production of phage particles as described (Scott & Smith (1990) Science 249, 386-390). For the assay, 1 μg p38α MAP kinase is coated onto MaxiSorp™ polystyrene plates (Nunc™ brand, Fisher Scientific, Loughborough, U.K.) in 100 μl PBS for 1 hour at room temperature and then washed once with PBS followed by blocking with 2% BSA in PBS for 1 hour at room temperature. 100 μl of phage supernatant is added per well and incubated for 1 hour at room temperature. The plates are washed four times with PBS/Tween20 and twice with PBS. The horseradish peroxidase-conjugated (HRP) anti-M13 secondary antibody (GE Healthcare U.K. Ltd., Chalfont St. Giles, U.K.) is diluted 1:5000 in 2% BSA in PBS and incubated for an hour at room temperature followed by washing as above. ELISA assays are performed as described (McGregor & Robins (2001) Anal. Biochem. 294, 108-117). The assay was developed with SureBlue TMB peroxidase substrate (Insight Biotechnology, Middlesex, U.K.) and read at 450 nm. The peptides that bound p38α MAP kinase are tested for specificity against biotinylated p38α. MAP kinase and streptavidin and then against β-galactosidase, BcIX, anti-FLAG M2 antibody, ovalbumin and lysozyme all coated at 0.5 μg in a streptavidin coated plate (StreptaWell; Roche Diagnostics Ltd., Burgess Hill, U.K.) using the same ELISA conditions as described above. This method can be used to establish direct specific peptide binding to p38α. MAP kinase, and binding is indicated by an increased colorimetric signal over control background.

It is particularly preferred if the compound is one which competes for binding to p38α MAP kinase. Whether or not a compound is one which can compete for binding to p38α MAP kinase can be determined as follows.

The N-acetylated peptide may be labelled fluorescently (i.e. with a fluorophore) and is bound to p38α MAP kinase. Displacement of this peptide by a compound is indicative of binding of that compound to p38α MAP kinase and can be measured by fluorescence polarization. Fluorescence polarization is an empirical fluorescence detection technique that measures the vertical and horizontal components of fluorescence emission created using plane polarized excitation. Polarization values (measured in mP units) for any fluorophore-labelled complex are inversely related to the speed of molecular rotation of that complex. Since molecular rotation is, in turn, inversely related to its molecular volume, the fluorescence-labelled peptide will possess a higher polarization value when it interacts with any molecule large enough to slow its rate of molecular rotation (p38α MAP kinase in this case). The magnitude of the polarization signal is thus used to quantitatively determine the extent of fluorescence-labelled peptide binding without the need for any filtration or wash separation step. The principle of this displacement binding assay is competition between the fluorescently labelled peptide and the unlabelled compound for binding to the p38α MAP kinase docking site, using a fluorescence polarization detection method. Binding of unlabelled compound causes displacement of the fluorescently labelled peptide and a loss of mP signal. Any loss of signal indicates displacement. A large loss of signal indicates greater displacement than a small loss of signal which indicates lower displacement. Any number of fluorescent polarisation readers can be used to measure displacement.

It is particularly preferred if the compound of the invention is one which binds to and inhibits p38αMAP kinase, but does not substantially bind to and inhibit any of Jnnk, PKCd, p38b, AMPK, AurA, GK3b, RAF1, JNK3, VEGF, p38g, p38d, PKCb, PKCa, PDHK2, PDK1, MKK6, p27 KIP, Cdk2 KIP, Prak KIP, Chk1 KIP, Egfr KIP, Kdr KIP, EGF KIP, Zap 70 KIP, IGFR KIP, Src KIP, Fak KIP, Jak3 KIP, Akt CIRA, and Mek CCEK. Commercial services are available to determine whether a compound inhibits these and other protein kinases using standard methods. For example, Merck Millipore's Kinase Profiler™ may be used and version 54 of its Service Assay protocols.

The compounds of the invention may have an IC₅₀ value of >1 μM, and more preferably >5 μM or >10 μM for at least one if not all of the enzymes listed to which it does not substantially bind.

The compounds of the invention may have an IC₅₀<1 μM against human p38α MAP kinase. The compounds of the invention may have an IC₅₀<0.1 μM, preferably <0.05 μM, more preferably <0.01 μM, and still more preferably <0.001 μM, as measured using the following methodology. IC₅₀ is calculated by testing the degree of inhibition of a fixed concentration of p38α MAP kinase using increasing concentrations of the compounds of the invention. When plotted on a log scale with the degree inhibition on the y axis and peptide concentration on the x-axis this will normally provide a sigmoid curve from 0% inhibition at the 0 or lowest concentration up to 100% inhibition at the highest doses. The point at which 50% inhibition is achieved is called the inhibitory concentration 50 or IC₅₀ and is used to establish the potency of a compound at a fixed concentration of enzyme.

The compounds of the invention may display selectivity for p38α MAP kinase, which may be due in part to the compounds of the invention binding to a site on p38α MAP kinase which is not present in other protein kinases, and not to the ATP binding pocket.

The site is believed to be conserved between human p38α MAP kinase and canine p38α MAP kinase. Inhibition of p38α MAP kinase by the binding of a compound at an unusual, and possibly unique, binding pocket offers the much greater likelihood of selective inhibition of p38α MAP kinase and therefore avoiding off target side effects and toxicity.

According to a further aspect of the invention there is provided a method of inhibiting p38α MAP kinase, said method comprising contacting the said kinase with a compound of the invention. Typically, the method is carried out in vitro.

Thus, the compounds of the invention may be useful as reagents for analysing p38α MAP kinase.

According to a further aspect of the invention, there is provided a kit of parts for analysing p38α MAP kinase, said kit comprising a compound of the invention and a substrate for p38α MAP kinase. Suitable substrates for p38α MAP kinase include myelin basic protein (MBP), MK2/MAPKAPK2, MNK-1, PRAK, and MSK1. MBP is a preferred substrate.

Also described is the use of a compound of the invention for inhibiting p38α MAP kinase.

Inhibition of p38α MAP kinase is known to be useful therapeutically, particularly in the treatment of proliferative disorders (e.g. cancer), autoimmune disorders, inflammatory-disorders, metabolic disorders, and neurological disorders. Therefore, according to a further aspect of the invention there is provided the compounds as herein described for use in the manufacture of a medicament, e.g. to treat an individual. Thus, there is particularly provided the compounds as herein described for use in the manufacture of a medicament for the treatment of proliferative disorders (e.g. cancer), autoimmune disorders, inflammatory-disorders, metabolic disorders, and neurological disorders. The individual may be a human or a non-human animal, such as a non-human mammal. Because of the degree of amino acid sequence similarity (99.4% identity) to human p38α MAP kinase (MK14_HUMAN SEQ ID No: 18) and canine p38α MAP kinase (MK14_CANFA SEQ ID No: 19), the compounds are particularly suited for the treatment of man and dogs. The compounds may also be useful for treating other animals which have a p38α MAP kinase with substantial sequence similarity (e.g. >90% identity) to the human enzyme sequence.

The compounds of the invention may be used either alone or in combination with other therapeutic compounds. Thus, according to a further aspect of the invention there is provided a composition comprising a compound of the invention in combination with one or more other therapeutic compounds, such as anti-inflammatory or anti-infective (e.g. anti-bacterial or anti-viral) or anti-proliferative compounds.

The therapeutic composition of the invention may include a pharmaceutically acceptable adjuvant, diluent or carrier. The excipient(s) and carrier(s) must be “acceptable” in the sense of being compatible with the compound and not deleterious to the recipients thereof. Suitable pharmaceutical adjuvants, diluents or carriers may be selected as known in the art depending on the route of administration for the compound or composition and whether the compound or composition is for human or veterinary use.

The compounds of the invention or a therapeutic composition thereof may be administered by any conventional method known in the art. The administration may consist of a single dose or administration or a plurality of doses or administrations over a period of time.

The therapeutic composition may be in a unit dosage containing a daily dose or unit, daily sub-dose or an appropriate fraction thereof, of the compound of the invention as active ingredient.

The compounds of the invention will normally be administered topically or orally or inhaled or by any parenteral route, in the form of a pharmaceutical formulation, optionally in the form of a non-toxic organic, or inorganic, acid, or base, addition salt, in a pharmaceutically acceptable form. Depending upon the disorder and individual to be treated, as well as the route of administration, the formulations may be administered at varying doses.

Thus, the compounds of the invention or the therapeutic composition of the invention can be administered orally, buccally, intranasally, intra-occularly, inhaled, topically, rectally or sublingually in the form of tablets, capsules, ovules, elixirs, solutions, sprays, gels or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds may be combined with various sweetening or flavouring agents, colouring matter or dyes, with emulsifying and/or suspending agents and with diluents such as water, ethanol, propylene glycol and glycerin, and combinations thereof.

The compounds of the invention or the therapeutic compositions of the invention may also be administered parenterally, for example, intravenously, intra-arterially, intraperitoneally, intrathecally, intraventricularly, intrasternally, intrathoracically, intracranially, intra-muscularly, subcutaneously or intradermally, or they may be administered by infusion techniques. They may be used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The compounds of the invention or the therapeutic compositions of the invention may also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound described and a suitable powder base such as lactose or starch.

Aerosol or dry powder formulations may be arranged so that each metered dose or “puff” contains at least 1 μg of the compound of the invention or the therapeutic composition of the invention for delivery to the patient. It will be appreciated that the overall daily dose with an aerosol will vary from patient to patient, and may be administered in a single dose or, more usually, in divided doses throughout the day.

Alternatively, the compounds of the invention or the therapeutic compositions of the invention may be administered in the form of a suppository or pessary, or they may be applied topically in the form of a lotion, solution, gel, cream, ointment or dusting powder. The compounds of the invention or the therapeutic compositions of the invention may also be transdermally administered, for example, by the use of a skin patch. They may also be administered by the ocular route, particularly for treating diseases of the eye.

For ophthalmic use, the compounds of the invention or the therapeutic compositions of the invention may be formulated using nanoparticle systems or as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention or the therapeutic compositions of the invention may be formulated using nanoparticle systems as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable spray, gel, lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. The compounds may also be formulated with nanoparticles which have proven tissue and cell penetration qualities.

Furthermore, for application topically, the compounds of the invention or the therapeutic compositions of the invention (whether alone or in combination with other active ingredients or materials) may be applied to, or impregnated into, a wound dressing so as to provide a dressing which can be used to treat a proliferative disorder (e.g. cancer), an autoimmune disorder, an inflammatory-disorder, a metabolic disorder, and a neurological disorder or improve wound healing in an individual.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

The compounds of the invention or the therapeutic compositions of the invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The compounds of the invention or the therapeutic compositions of the invention may be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of peptides. A device which delivers a pulsed electric field to cells increases the permeability of the cell membranes to the drug, resulting in a significant enhancement of intracellular drug delivery. Thus, electroporation therapy (EPT) systems may be employed for the administration of the compounds of the invention or the therapeutic compositions of the invention.

Peptides can be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter. Thus, electroincorporation may be employed for the administration of the compounds of the invention or the therapeutic compositions of the invention.

An alternative method of peptide delivery is the ReGel injectable system that is thermosensitive. Below body temperature, ReGel is an injectable liquid while at body temperature it immediately forms a gel reservoir that slowly erodes and dissolves into known, safe, biodegradable polymers. The active agent is delivered over time as the biopolymers dissolve. Thus, the ReGel injectable system may be employed for the administration of the compounds of the invention or the therapeutic compositions of the invention.

The compounds of the invention or the therapeutic compositions of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient.

The compounds of the invention or the therapeutic compositions of the invention can be introduced to cells by “transfection agents” including, but not limited to, polymers, liposomes, poly(lactic-co-glycolic acid) (PGLA, PLA), lipid nanoparticles, gold nanoparticles and other nanoparticles. As a class, these agents are capable of carrying compounds across the plasma membrane. These systems allow direct targeting of oligopeptides to the cytoplasm, and may be non-cell type specific or made cell-type specific and highly efficient.

The amount of the compound to be administered to the individual may be determined by the medical practitioner (doctor or vet) and will depend on whether the individual is a human or animal (and the type of animal), the condition to be treated and the route and frequency of administration. Conveniently, the compound may be administered to the skin in carrier formulation so that from 1 μg to 100 mg in carrier formulation is delivered per 2 cm² of area of inflammation, for example 100 μg, 1 mg, 5 mg, 10 mg or 50 mg.

Also described is a method for treating a proliferative (e.g. cancer), autoimmune disorder, an inflammatory-disorder, a metabolic disorder, and a neurological disorder in an individual, the method comprising administering to the individual a compound or a composition as described. The individual may be administered the compound described and the other therapeutic compound either sequentially in any order or simultaneously.

According to this aspect of the invention the method may comprise the treatment of an inflammatory-disorder; said treatment may comprise the treatment of burns and/or preventing scar formation; or the treatment of atopic dermatitis, the prevention of atopic dermatitis recurrence and delay ageing of the skin.

According to a further aspect of the invention there is provided a compound, or a therapeutic composition as herein described, for use in treating a proliferative disorder (e.g. cancer), an autoimmune disorder, an inflammatory-disorder, a metabolic disorder, and a neurological disorder in an individual.

According to this aspect of the invention the compound, or a therapeutic composition, for use in treating an inflammatory-disorder, may comprise use in the treatment of burns and/or preventing scar formation; or use in the treatment of atopic dermatitis.

According to a further aspect of the invention there is provided the use of the compound of the invention or a therapeutic composition of the invention, in the manufacture of a medicament, e.g. for treating or preventing a proliferative disorder (e.g. cancer), an autoimmune disorder, an inflammatory-disorder, a metabolic disorder, senescence, and a neurological disorder in an individual.

Proliferative disorders (e.g. cancer) include, but shall not be limited to, cancers such as breast, prostate, hematological malignancies (e.g. CML, AML, lymphomas), head and neck, colon, bladder, skin, prostatic adenocarcinoma, lung, cervical, and lymphomas.

Autoimmune disorders include, but shall not be limited to, autoimmune diseases include, but are not limited to, glomerulonephritis, Goodspature's syndrome, necrotizing vasculitis, lymphadenitis, periarteritis nodosa, epidermolysis bullosa, systemic lupus erythematosis, rheumatoid, arthritis, psoriatic arthritis, eosinophilic oesophagitis, systemic lupus erythematosis, psoriasis, ulcerative colitis, systemic sclerosis, dermatomyositis/polymyositis, anti-phospholipid antibody syndrome, scleroderma, perphigus vulgaris, ANCA-associated vasculitis (e.g. Wegener's granulomatosis, microscopic polyangiitis), uveitis, Sjogren's syndrome, Crohn's disease, Reiter's syndrome, ankylosing spondylitis, Lyme arthritis, Guillain-Barre syndrome, Hashimoto's thyroiditis, and cardiomyopathy.

Inflammatory-disorders, include, but shall not be limited to, inflammatory diseases, such as, atherosclerosis, arteriosclerosis, stenosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, post-operative scarring, post-traumatic scarring, burns, giant cell arteritis, progressive systemic sclerosis (scleroderma), ankylosing spondylitis, senescence, polymyositis, dermatomyosifis, pemphigus, pemphigoid, diabetes (e.g. Type I), myasthenia gravis, Hashimoto's thyroditis, Graves' disease, Goodpasture's disease, mixed connective tissue disease, sclerosing cholangitis, inflammatory bowel disease, Crohn's disease, ulcerative colitis, pernicious anemia, inflammatory dermatoses, atopic dermatitis, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, inflammatory pain, hepatitis, delayed-type hypersensitivity reactions (e.g. poison ivy dermatitis), pneumonia, respiratory tract inflammation, Adult Respiratory Distress Syndrome (ARDS), ventilator associated pneumonitis (VAP), coronavirus-pneumonitis, encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, graft-versus-host (GvH) rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomylitis, optic neuritis, temporal arteritis, transverse myelitis, necrotizing fascilitis, and necrotizing enterocolitis. An inflammatory disease can be either an acute or chronic inflammatory condition and can result from infections or non-infectious causes.

The inflammatory disorders to be treated include those in which the inflammatory pathway containing p38α MAP kinase plays a role in its pathology. Inflammatory disorders for treatment or prevention by the compounds described include, but are not limited to, inflammatory disorders of the skin such as atopic dermatitis, psoriasis, acne vulgaris, epidermolysis bullosa, dermal scarring and senescence. Activation of MAP Kinase p38α may also play a fundamental role in other disorders involving inflammation including, constrictive obstructive pulmonary disease, asthma, inflammatory bowel disease, atherosclerosis, cancer and rheumatoid arthritis, and these disorders may also be treated using the compounds described. The inflammatory condition which is treated may be a temporary, regular or permanent condition experienced by an individual.

Metabolic disorders include, but shall not be limited to, metabolic diseases such as obesity, as well as related disorders such as eating disorder, cachexia, diabetes mellitus, hypertension, coronary heart disease, peripheral vascular disease, hypercholesterolemia, dyslipidemia, osteoarthritis, gallstones, and sleep apnea.

Neurological disorders include, but shall not be limited to, neurodegenerative disorders such as Alzheimer's disease, dementia spectrum disorders (e.g. frontotemporal dementia (FTD), dementia of Lewy bodies (DLB), Creutzfeldt-Jakob disease (CJD)), autism, or autism spectrum disorders (e.g. Asperger syndrome or Mendelsohnn's Syndrome), myasthenia gravis.

According to a further aspect of the invention there is provided a method for identifying a molecule that binds to p38α MAP kinase, the method comprising determining whether the molecule binds to p38α MAP kinase at the position that a compound of the invention binds to p38α MAP kinase.

The method may be used to identify molecules which are useful for selectively inhibiting p38α MAP kinase, and which are useful in anti-inflammatory or autoimmune therapy. There are various ways of identifying whether a test molecule binds to p38α MAP kinase at the same position as the compounds of the invention. One way is to conduct biochemical competition assays between the test molecule and a compound of the invention to determine whether the test molecule is able to displace the peptide with compound of the invention from its binding site on p38α MAP kinase, for example by using the fluorescence polarization method described herein. The compound of the invention may be labelled with a detectable, such as fluorescent label. An affinity-based assay, or an enzyme linked immunoassay (ELISA) are used. Another way is to use computational chemistry. High resolution structural information produced via X-ray crystallisation or 2-dimensional nuclear magnetic resonance may be used to build a precise computational model p38 MAP kinase.

The invention will now be described in a non-limiting way by reference to the following Figures, in which the peptides are referred to in the following manner:

JEL0305 is:
Val-Ser-Arg-Ala-Thr-Arg-Ile-Phe-Lys-Leu-Ser-Gln-
Leu-Arg-Gly-Gln-Cys-Ala;
and
JEL2603 is:
Ser-Arg-Ala-Leu-Lys-Ile-Phe-Leu-Ala-Thr-Arg-Lys-
Asp-Arg-Ser-Pro-Trp-His is;
and
JEL2603 is:
His-Lys-Ser-Arg-Ala-Arg-Leu-Ile-Phe-His-Glu-Gly-
Pro-Cys-Gly-Val-Arg-Arg;
and
JEL0802 is:
Ser-Arg-Ala-Leu-Leu-Ile-Phe-Gln-Lys-Ile is;

In the following set of figures, differences were statistically significant for *p<0.05, **p<0.01, ***p<0.001 and ****p<0.0001 (N=2).

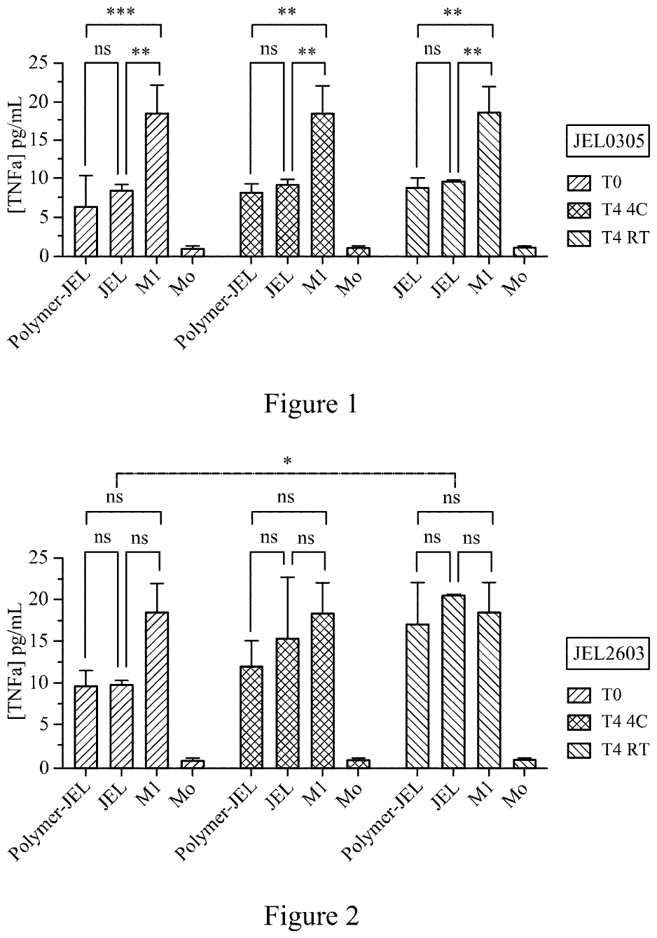

FIG. 1 illustrates activity of JEL0305 at baseline and at 4 weeks, at two storage conditions (4° C. and room temperature (RT)). Both show similar activity to T0 indicating the biological activity of JEL0305 is stable when stored either at RT or 4° C. for both the peptide and the polymer formulation;

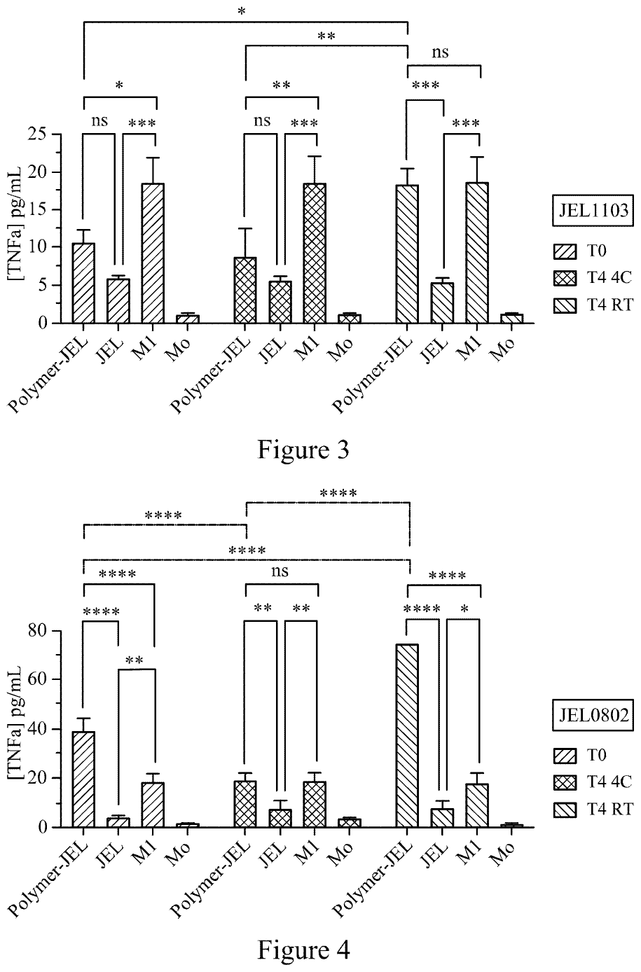

FIG. 2 illustrates JEL2603 which indicates there is some diminution of anti-inflammatory activity between T0 and 4 weeks when stored at 4° C. At RT activity is lost at 4 weeks for both the peptide and the polymer formulation;

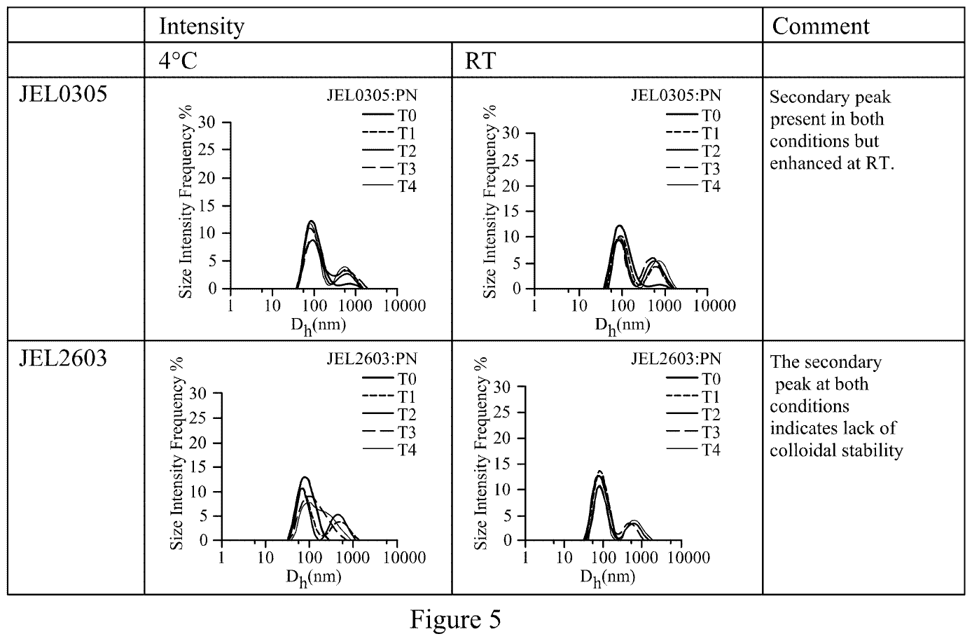

FIG. 3 illustrates JEL1103 that at 4° C. and RT the activity of JEL1103 peptide is maintained but the JEL1103-polymer formulation loses activity when stored at RT indicating a deficiency in this polymer formulation;

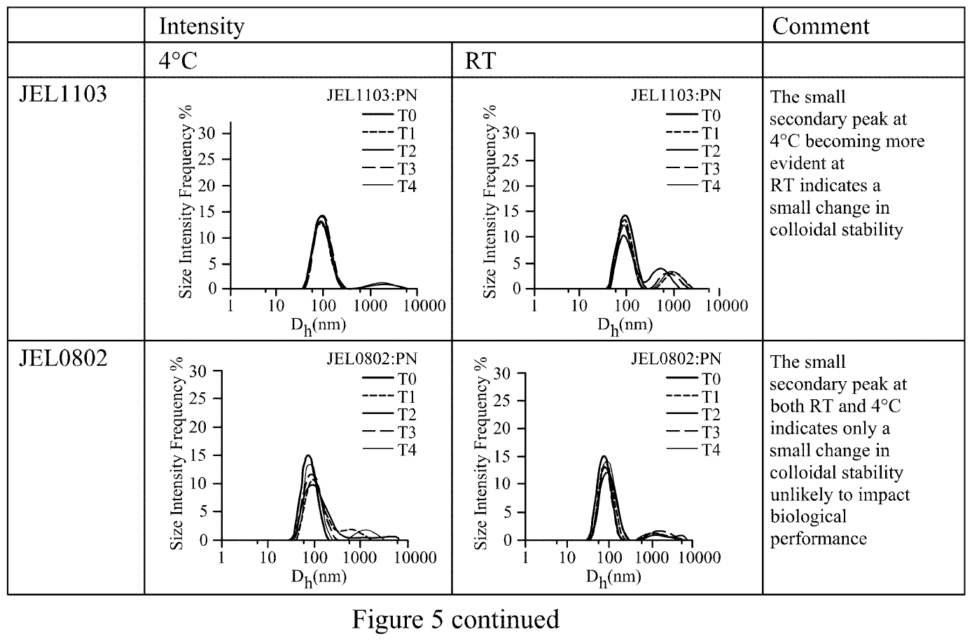

FIG. 4 illustrates that JEL0802 shows activity at T0 at 4 weeks at 4° C. and RT for the peptide (JEL0802) alone. The peptide-polymer formulation adds no benefit but show pro-inflammatory effects of the polymer;

In FIGS. 1-4:
M0 means non-activated macrophages
M1 means pro-inflammatory macrophage after stimulation with LPS.
Polymer-JEL means combine peptide (JEL) with polymer transfection agent.
JEL means peptide on its own

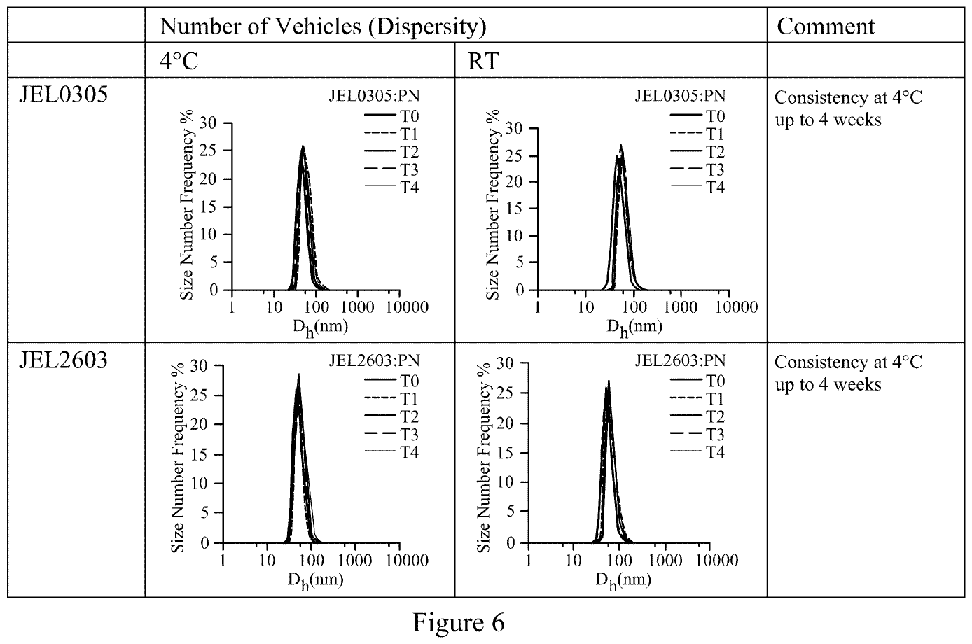

FIG. 5 illustrates Particle Intensity vs Size at T0, T1, T2, T3, and T4 weeks and shows a secondary peak due to the polymer; and

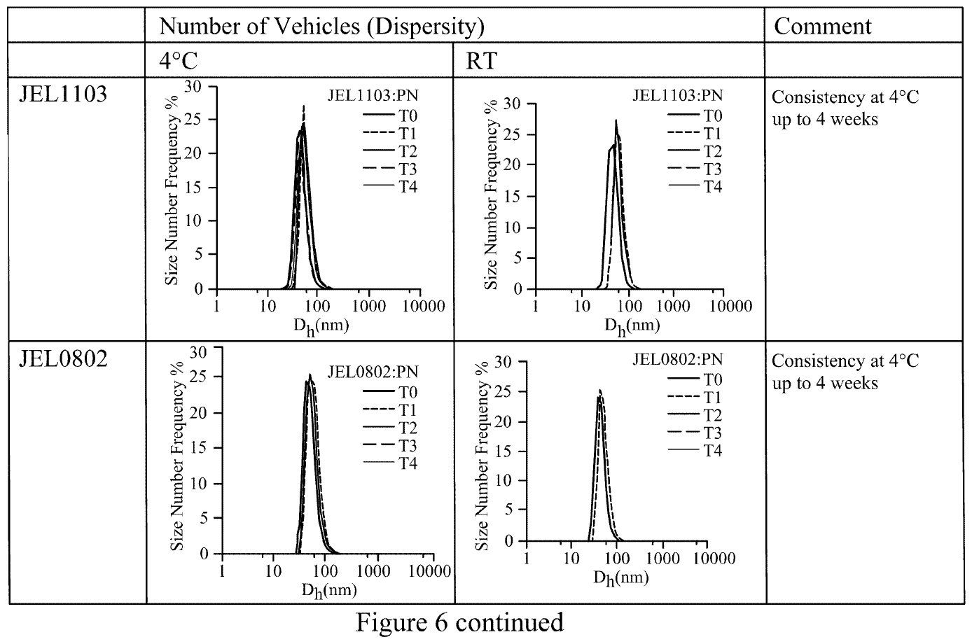

FIG. 6 illustrates Particle Number vs Size at T0, T1, T2, T3, and T4 weeks and indicates consistency of the nanoparticle formulation at 4 weeks, both at 4° C. and RT.

Each peptide was encapsulated in a propriety nanoparticle formulation, purified, characterised for physical-chemical properties and assessed for anti-inflammatory biological activity in vitro. Other transfection methods have been used including but not limited to proprietary polymers, PGLA, liposomes and lipid nanoparticles. Optimised formulations were prepared and stored at two conditions (room temperature and 4° C.) and a stability study undertaken up to 4 weeks. The key stability indicating parameters tested were assay and anti-inflammatory activity by ELISA. In addition, vesicular stability was assessed.

### Vesicular Stability

The FDA guidance for the formal stability of drug product nanoparticles (lipid nanoparticles are the main reference) requires an extensive evaluation of the integrity of the vesicle. This may be done through the measurement of vesicular stability. Vesicular stability provides a measure of the integrity of the vesicle membrane and is useful in development work to understand functionality.

Two Measures of Vesicular Stability were Assessed
The number vs size graph gives equal weighting to each particle. The size of a peak is directly proportional to the number of particles corresponding to that peak.
The intensity vs size graph gives weighting to a particle based on the intensity of scattered light i.e. the bigger the particle, the higher the intensity of the scattered light.

The vesicular stability was determined using Dynamic Light Scattering (DLS).

### In Vitro Anti-Inflammatory Activity (ELISA)

The anti-inflammatory response of each free peptide and each peptide-polymer formulation was determined using TNFα as a marker. The anti-inflammatory bioactivity was assessed using enzyme-linked immunosorbent assay (ELISA) for quantitative detection of human TNFα in activated M1-macrophages. THP1 human monocytes were differentiated into macrophages (M0), which were then activated (overnight) with lipopolysaccharide (LPS) into M1-macrophages. LPS prompts the abundant production of many pro-inflammatory cytokines from macrophages, including TNF-α, which actively contributes to the inflammatory response.

At the T0 and T4 (after 4 weeks storage at RT or 4° C.) time points, each JEL peptide and each JEL-polymer formulation was incubated in previously LPS-activated M1-macrophages for 24 h at 37° C. After incubation, supernatants were collected, centrifuged and stored at −80° C. in readiness for the enzyme-linked immunosorbent assay (ELISA).

Each supernatant sample was defrosted and the ELISA assay undertaken according to the Invitrogen protocol.

FIGS. 1-4 show the concentrations of human TNFα detected by ELISA after incubation with each free and polymer loaded peptide at a concentration of 100 ng/mL.

### Skimune® Skin Explant Assays to Model a Graft Versus Host Disease (GvHD) and Cytokine Responses

Using a HLA (human leucocyte antigen) mismatched GvHD model which gives rise to grade III to IV Graft versus Host reactions, all tests using an LNP formulation of the compound of the invention (JEL0305, JEL2603, JEL1103 or JEL0802) and positive control assays showed Grade III reactions with no inhibition of the response being observed when the compound was added throughout the mixed lymphocyte reaction and spiked into the skin explant (Skimune®) assays.

Cytokine levels from the Skimune® assays were measured and found to contain very high levels of pro-inflammatory cytokines including Th1 (IFNγ, TNFα, IL-1β) and Th2 cytokines (IL-13 and IL-4). This result explains the Grade III responses in the Skimune® and suggests a response of the lipid NP formulation due to an excessive amount of lipid especially at the higher concentrations. Formulation of the peptide with a different nanoparticle formulation, a lower lipid dose or a polymer, for example, would suppress the effects of the lipid and allow the peptide to show its efficacy (as shown in the T cell proliferation assays) currently masked by the formulation.

### Atopic Dermatitis (AD) Model

The AD phenotype was assessed by measuring the intensity of the “white space” within the biopsies—this was increased from 20% in the media alone control to 80% on AD induction and reduced with Clobetasol (a topical corticosteroid) to 40%. All concentrations of the compound (JEL0305, JEL2603, JEL1103 or JEL0802) reduced the white space by between 50 and 60% as a similar trend to Clobetasol. Three further donors (ALC1373, ALC1366 and ALC1365) were used to develop atopic dermatitis after treatment with activated PBMC and a cytokine cocktail model and assessed phenotypically as well as by biomarker (Filaggrin, Involucrin and TSLP) by immunohistochemistry. For 2 donors (ALC1366 and ALC1365) Filaggrin and Involucrin were reduced compared non-AD induced skin biopsies in media alone. This is the expected response for AD induction. In the presence of the control drug Clobetasol the expression of Filaggrin and Involucrin was increased due to the drug reversing the AD phenotype. The compound (JEL0305, JEL2603, JEL1103 or JEL0802) at all concentrations (0.1 μg/ml, 1.0 μg/ml and 2.5 μg/ml) showed a similar increase in expression which was paralleled by a phenotype (“white space”) analysis showing an improved and reversal of damage in the treated samples. But this was not as apparent as Clobetasol more than likely due to the lipid formulation causing an upregulation of cytokines. The third donor ALC1373 was less conclusive although some improvement of the phenotype was observed with 0.1 and 1.0 μg/ml of the compound (JEL0305, JEL2603, JEL1103 or JEL0802) which appeared to be an improvement above the control drug Clobetasol. Filaggrin results were inconclusive.

TSLP expression for all donors was inconclusive.

The main overall conclusion is that the compound of the invention (JEL0305, JEL2603, JEL1103 or JEL0802), at the current concentrations and formulation causes significant increases in pro-inflammatory cytokines which mask the effects of the peptide when tested for inhibitory properties in “complex “GvHD type assays. However, when the compound (JEL0305, JEL2603, JEL1103 or JEL0802) was tested in a less complex tissue model of Atopic Dermatitis it demonstrated a similar response to that of a known drug Clobetasol. The results demonstrate that the compound (JEL0305, JEL2603, JEL1103 or JEL0802) has potential as an immunomodulator in Atopic Dermatitis.

1. A compound comprising a sequence of at least 3 amino acids wherein the sequence of 3 amino acids comprises the sequence Ser-Arg-Ala;
and variants and derivatives thereof,
provided that the amino acid is not:
His-Lys-Ser-Arg-Ala-Leu-Leu-Ile-Phe-Gln-Lys-Ile-
Met-Trp-Leu-Arg-Arg-Gln.

2. A compound according to claim 1 wherein the compound comprises a sequence of 7 amino acids comprises Ser-Arg-Ala-A-B-Ile-Phe, wherein A and B, which may be the same or different, may be any amino acid moiety;
and variants and derivatives thereof;
provided that the amino acid is not:
His-Lys-Ser-Arg-Ala-Leu-Leu-Ile-Phe-Gln-Lys-Ile-
Met-Trp-Leu-Arg-Arg-Gln.

3. A compound according to claim 1 wherein the compound is selected from the group comprising the amino acids:
Ser-Arg-Ala-Leu-Lys-Ile-Phe-Leu-Ala-Thr-Arg-Lys-
Asp-Arg-Ser-Pro-Trp-His;
His-Lys-Ser-Arg-Ala-Arg-Leu-Ile-Phe-His-Glu-Gly-
Pro-Cys-Gly-Val-Arg-Arg,Ser-Arg-Ala-Leu-Leu-Ile-
Phe-Gln-Lys-Ile;
and
Val-Ser-Arg-Ala-Thr-Arg-Ile-Phe-Lys-Leu-Ser-Gln-
Leu-Arg-Gly-Gln-Cys-Ala;
and variants and derivatives thereof.

4. A compound according to claim 1 wherein the compounds comprise variants comprising larger peptides with additional amino acids, in which the peptides of claim 1 form only part of the variant peptide.

5. A compound according to claim 1 wherein in the variant peptides the additional amino acids are naturally occurring amino acids.

6. A compound according to claim 1 wherein in the variant peptides the additional amino acids are non-natural amino acids, such as allylalanine and diphenylalanine.

7. A compound according to claim 1 which has a molecular weight of less than 30,000 daltons.

8. A compound according to claim 1 wherein the amino acids are joined by peptide (—CO—NH—) linkages.

9. A compound according to claim 1 which contains at least one non-peptide linkage between the amino acids.

10. A compound according to claim 1 wherein the compound comprises the amino acid sequence:
Ser-Arg-Ala-Leu-Lys-Ile-Phe-Leu-Ala-Thr-Arg-Lys-
Asp-Arg-Ser-Pro-Trp-His;
and variants and derivatives thereof.

11. A compound according to claim 1 wherein the compound comprises the amino acid sequence:
His-Lys-Ser-Arg-Ala-Arg-Leu-Ile-Phe-His-Glu-Gly-
Pro-Cys-Gly-Val-Arg-Arg;
and variants and derivatives thereof.

12. A compound according to claim 1 wherein the compound comprises the amino acid sequence:
Ser-Arg-Ala-Leu-Leu-Ile-Phe-Gln-Lys-Ile;
and variants and derivatives thereof.

13. A compound according to claim 1 wherein the compound comprises the amino acid sequence:
Val-Ser-Arg-Ala-Thr-Arg-Ile-Phe-Lys-Leu-Ser-Gln-
Leu-Arg-Gly-Gln-Cys-Ala;
and variants and derivatives thereof.

14. (canceled)

15. A compound according to claim 1 wherein some or all of the amino acids in the peptide are N-acetylated.

16.-21. (canceled)

22. A method of inhibiting p38α MAP kinase, said method comprising contacting the said kinase with a compound comprising a sequence of at least 3 amino acids wherein the sequence of 3 amino acids comprises the sequence Ser-Arg-Ala;
and variants and derivatives thereof;
provided that the amino acid is not:
His-Lys-Ser-Arg-Ala-Leu-Leu-Ile-Phe-Gln-Lys-Ile-
Met-Trp-Leu-Arg-Arg-Gln.

23. A method according to claim 22 wherein the compound comprises a sequence of at least 7 amino acids wherein the sequence of 7 amino acids comprises Ser-Arg-Ala-A-B-Ile-Phe, wherein A and B, which may be the same or different, may be any amino acid moiety;
and variants and derivatives thereof;
provided that the amino acid is not:
His-Lys-Ser-Arg-Ala-Leu-Leu-Ile-Phe-Gln-Lys-Ile-
Met-Trp-Leu-Arg-Arg-Gln.

24. A method according to claim 22 wherein the compound is selected from the group comprising the amino acids:
Ser-Arg-Ala-Leu-Lys-Ile-Phe-Leu-Ala-Thr-Arg-Lys-
Asp-Arg-Ser-Pro-Trp-His;
His-Lys-Ser-Arg-Ala-Arg-Leu-Ile-Phe-His-Glu-Gly-
Pro-Cys-Gly-Val-Arg-Arg,Ser-Arg-Ala-Leu-Leu-Ile-
Phe-Gln-Lys-Ile;
and
Val-Ser-Arg-Ala-Thr-Arg-Ile-Phe-Lys-Leu-Ser-Gln-
Leu-Arg-Gly-Gln-Cys-Ala;
and variants and derivatives thereof.

25. A method according to claim 22 which comprises treatment of a proliferative (e.g. cancer), autoimmune disorder, an inflammatory-disorder, a metabolic disorder, and a neurological disorder.

26. A method according to claim 25 wherein the treatment of an inflammatory-disorder comprises the treatment of burns and/or preventing scar formation.

27. A method according to claim 25 wherein the treatment of an inflammatory-disorder comprises the treatment of atopic dermatitis, the prevention of atopic dermatitis recurrence and delay ageing of the skin.

28.-36. (canceled)

37. A kit of parts for analysing p38α MAP kinase, said kit comprising a compound comprising a sequence of at least 3 amino acids wherein the sequence of 3 amino acids comprises the sequence Ser-Arg-Ala;
and variants and derivatives thereof,
provided that the amino acid is not:
His-Lys-Ser-Arg-Ala-Leu-Leu-Ile-Phe-Gln-Lys-Ile-
Met-Trp-Leu-Arg-Arg-Gln.

38. (canceled)

39. (canceled)

40. A composition comprising a compound comprising a sequence of at least 3 amino acids wherein the sequence of 3 amino acids comprises the sequence Ser-Arg-Ala;
and variants and derivatives thereof,
in combination with one or more other therapeutic compounds;
provided that the amino acid is not:
His-Lys-Ser-Arg-Ala-Leu-Leu-Ile-Phe-Gln-Lys-Ile-
Met-Trp-Leu-Arg-Arg-Gln.

41. (canceled)

42. (canceled)

43. A composition according to claim 40 wherein the one or more other therapeutic compounds is an anti-inflammatory, anti-infective (e.g. anti-bacterial or anti-viral), autoimmune suppressant, anti-metabolic, anti-neurological or anti-proliferative compound.

44. A composition according to claim 40 wherein the composition includes a pharmaceutically acceptable adjuvant, diluent or carrier.

45. A compound according to claim 1, for use in treating a proliferative (e.g. cancer), autoimmune disorder, an inflammatory-disorder, a metabolic disorder, and a neurological disorder in an individual.

46. A compound according to claim 45 wherein the treatment of an inflammatory-disorder comprises the treatment of burns and/or preventing scar formation.

47. A compound according to claim 45 wherein the treatment of an inflammatory-disorder comprises the treatment of atopic dermatitis, the prevention of atopic dermatitis recurrence and delay ageing of the skin.

48. (canceled)

49. A composition according to claim 40 for use in treating a proliferative (e.g. cancer), autoimmune disorder, an inflammatory disorder, a metabolic disorder, and a neurological disorder in an individual.
